(19) United States
(12) Patent Application Publication
(10) Pub. No.: US 2013/0095135 A1
(43) Pub. Date: Apr. 18, 2013

(54) PROCESS FOR REMOVING ADVENTITIOUS AGENTS DURING THE PRODUCTION OF A VIRUS IN CELL CULTURE

(71) Applicants: Francoise Marie Isabelle Collignon, Rixensart (BE); Isabelle Solange Lucie Knott, Rixensart (BE); Jean-Philippe Jules Jeanine Matheise, Rixensart (BE)

(72) Inventors: Francoise Marie Isabelle Collignon, Rixensart (BE); Isabelle Solange Lucie Knott, Rixensart (BE); Jean-Philippe Jules Jeanine Matheise, Rixensart (BE)

(73) Assignee: GlaxoSmithKline Biologicals S.A., Rixensart (BE)

(21) Appl. No.: 13/805,397

(22) PCT Filed: Jul. 6, 2011
(86) PCT No.: PCT/EP11/61446

§ 371 Date: Dec. 19, 2012

(30) Foreign Application Priority Data
Jul. 8, 2010 (GB) 1011502.0

(51) Int. Cl. C12N 7/02

(52) U.S. Cl. CPC C12N 7/02
USPC 424/215.1; 435/235.1; 435/239

(57) ABSTRACT

The present invention relates to improved processes for the production of viruses, in particular, viruses for use in medicine (for example vaccination or gene therapy).

### TECHNICAL FIELD

The present invention relates to improved processes for the production of viruses, in particular, viruses for use in medicine (for example vaccination or gene therapy).

### BACKGROUND TO THE INVENTION

One of the ways developed recently to produce medicines comprising a virus (e.g. an immunogenic composition or a vaccine) relies on cell culture systems, in particular mammalian cell cultures. Typically, those systems involve the infection of cells with a virus of interest and purification of the virus from the cells after a sufficient time for replication and production of the virus in the cells. While producing the virus on cells, other raw materials (e.g. tissue culture reagents, stabilizers) may be added to the virus at various stages of production. Thus, adventitious agents can potentially enter a viral product through any of these ingredients and contaminate said product. Viruses produced on cell substrates are particularly prone to this type of contamination. Adventitious agents, of which a major constituent are viruses, are known to contaminate biological materials such as cell lines.

### SUMMARY OF THE INVENTION

In a first aspect, the present invention provides a process for producing a virus of interest in cell culture, comprising contacting a population of cells with a solution comprising the virus of interest, wherein the cells are susceptible to infection with the virus of interest, characterised in that the contact time between the solution comprising the virus of interest and susceptible cells is inferior than or equal to 120 minutes.

In a second aspect, the present invention provides a process for producing a virus of interest comprising the steps of:
a) contacting a population of cells with a solution comprising the virus of interest for a period of time inferior than or equal to about 120 minutes, wherein the cells are susceptible to infection with the virus of interest,
b) removing the solution comprising the virus of interest from the cells, and
c) incubating the cells in a culture medium to produce a population of replicated virus of interest.

In a third aspect, the present invention provides a process for producing a virus of interest for use in medicine comprising the steps of:
a) contacting a population of cells with a solution comprising the virus of interest for a period of time inferior than or equal to about 120 minutes, wherein the cells are susceptible to infection with the virus of interest,
b) removing the solution comprising the virus of interest from the cells,
c) incubating the cells in a culture medium to produce a population of replicated virus of interest, and optionally
d) formulating the produced virus with a suitable pharmaceutical carrier

In a fourth aspect, the present invention provides a process for producing a virus of interest comprising the steps of:
a) contacting a population of cells with an initial solution comprising the virus of interest and at least one adventitious agent, wherein the cells are susceptible to infection with the virus of interest and the contact period of time is sufficient to permit infection of at least a subset of the population of cells with the virus of interest,
b) removing the solution comprising the virus of interest and the at least one adventitious agent from the cells,
c) incubating the cells in a culture medium to produce a population of replicated virus of interest, wherein the DNA content level of the at least one adventitious agent is reduced by at least 90% in the population of replicated virus of interest as compared to the level present in the initial solution comprising the virus of interest and the at least one adventitious agent.

In a fifth aspect, the present invention provides a process for removing and/or reducing the incidence of adventitious agents during the replication of a virus of interest, comprising the steps of:
i) inoculating susceptible cells with a viral inoculum comprising the virus of interest and one or more adventitious agents,
ii) incubating the inoculated cells, and
iii) washing said inoculated cell culture after less than 120 minutes after inoculation

In a further aspect, the present invention provides a process for removing adventitious agents during the production of a virus of interest on cell culture comprising the steps of:
a) contacting a population of cells with a solution comprising the virus of interest and at least one adventitious agent for a period of time inferior than or equal to about 120 minutes, wherein the cells are susceptible to infection by the virus of interest,
b) removing the solution comprising the virus of interest and the at least one adventitious agent from cells,
c) incubating the cells in a culture medium to produce a population of replicated virus of interest.

In a still further aspect, the present invention provides a process for removing an adventitious agent during the production of a virus of interest on cell culture comprising the steps of:
a) contacting a population of cells with an initial solution comprising the virus of interest and at least one adventitious agent, wherein the cells are susceptible to infection with the virus of interest and the contact period of time is sufficient to permit infection of at least a subset of the population of cells with the virus of interest,
b) removing the solution comprising the virus of interest and the at least one adventitious agent from the population of cells,
c) incubating the cells in a culture medium to produce a population of replicated virus of interest, wherein the at least one adventitious agent is removed or substantially removed from the population of replicated virus of interest as compared to the initial solution comprising the virus of interest and the at least one adventitious agent.

The invention also provides research, master and/or working viral seeds obtainable by the processes of the invention; as well as immunogenic compositions or vaccines produced from virus seeds of the invention.

The invention also provides virus suspensions or virus preparations obtainable by any of the processes described herein, vaccine/immunogenic compositions comprising said suspensions or preparations, and their use in medicine, in particular the prophylaxis and/or treatment of a disease.

### DETAILED DESCRIPTION OF THE INVENTION

Viruses display various physico-chemical properties, and thus their susceptibility to physical or chemical treatment varies. Typically, enveloped viruses are less resistant than non-enveloped viruses and it is well known that solvents can destroy enveloped viruses such as orthomyxoviridae or paramyxoviridae.

Methods to clear contaminating adventitious agents, in particular, adventitious viruses, from a virus culture suitable for use in medicine can often be difficult to define, especially when the adventitious virus is more resistant to physico-chemicals treatment than the virus of interest. When the virus of interest and the adventitious virus are able to replicate on the same cell culture, the methods for removing and/or reducing the incidence of the adventitious virus are more limited. A classical method to get rid of any adventitious agent is to clone by end-point dilution the virus of interest (for example, the vaccine virus). Anti-virals or inhibitors of the adventitious virus can be used when known, although the impact on the virus of interest is generally unknown. It is highly desirable to remove adventitious agents, when present, from cells used to produce a virus of interest to avoid and/or reduce any interference in the replication of the virus of interest, or remove adventitious agents from the viral suspension resulting from the multiplication and production of the virus of interest.

Viruses that are suitable for use in immunogenic compositions, such as vaccines and/or gene therapy, are grown in cell cultures that allow replication of the virus, i.e. the cells are susceptible to the viruses. Adventitious agents can contaminate cell cultures, the virus of interest inoculum used to inoculate said cells as well as any of the reagents used during viral replication and production (e.g. media). Although some contaminants may be harmless, it is particularly desirable to remove or to substantially reduce the incidence or presence of adventitious agents as they may interfere with growth of the virus of interest to be used in a vaccine and/or gene therapy. Similarly, for safety reasons and health regulatory concerns, it is preferable to have a final product, such as a vaccine comprising a virus, free or substantially free of any adventitious agent contamination. It is thus an object of the present invention to provide a method for removing adventitious agents when producing a virus on cell culture.

The present inventor(s) have demonstrated a means for removing and/or reducing the incidence or the presence of adventitious agents during replication/multiplication and production of a virus of interest in a susceptible cell culture. Surprisingly, the inventors observed that reducing the contact time between susceptible cells and a solution comprising a virus of interest resulted in the production of the virus of interest free or substantially free of adventitious agents.

The term “contact time” is well known in the art and as used herein means the period of time a viral inoculum is left in contact with cells after inoculation, i.e. before the viral inoculum is removed, or substantially removed, by washing (i.e. replacing the media on the cells with virus-free media 1, 2, 3, 4, 5 or more times, as appropriate). The reduced contact time may vary depending on the type of virus of interest to be produced. For example, the duration of the contact may be as short as the time required to get the virus adsorbed or attached to at least a subset of susceptible cells. The skilled person is able to monitor the attachment kinetic of a given virus to given cells, and determine thus the optimal virus contact time. The maximum duration of the virus contact time may be determined so that the amount of adventitious agents, when detectable in the virus of interest inoculum, is reduced by at least 50%, suitably by 90%, more suitably by 95% and even by 99% or 99.9% in the virus of interest preparation obtained according to processes of the invention. Accordingly, in some embodiments, in the processes of the invention susceptible cells are contacted with a solution comprising a virus of interest and at least one adventitious agent wherein the contact period of time is sufficient to permit infection, in particular to permit at least adsorption, of at least a subset of the population of cells with the virus of interest, and wherein the DNA content level of the adventitious agent is reduced by at least 90%, suitably at least 99% and more suitably 99.9% in the produced preparation of virus of interest, as compared with the level initially present in the solution comprising the virus of interest.

A suitable contact time in the processes of the invention is inferior than or equal to 120 minutes, for example less than about 90 minutes, about 60 minutes, about 45 minutes, about 30 minutes, 25 minutes, about 20 minutes, about 15 minutes, about 10 minutes, about 5 minutes, about 1 minute, or less than 45 seconds, in particular from about 5 seconds to about 1 hour, from about 10 seconds to about 55 minutes, from about 15 seconds to about 50 minutes from about 20 seconds to about 45 minutes, from about 30 seconds to about 40 minutes, from about 45 seconds to about 35 minutes or about 1 minute to about 30 minutes, for example from 10 to 30 seconds, from 20 seconds to 1 minute, from 10 seconds to about 30 minutes. In a particular embodiment of the invention, the contact time is at least 10 seconds. In further embodiments, the contact time is less than about 45 minutes, in particular less than about 35 minutes for example from about 5 seconds to about 1 hour, from about 10 seconds to about 55 minutes, from about 15 seconds to about 50 minutes from about 20 seconds to about 45 minutes, from about 30 seconds to about 40 minutes, from about 45 seconds to about 35 minutes or about 1 minute to about 30 minutes, for example from 10 to 30 seconds, from 20 seconds to 1 minute, from 10 seconds to about 30 minutes. Accordingly, in particular embodiments of the invention, the virus inoculum or virus solution is contacted with susceptible cells for a period of time ranging from 10 seconds to 120 minutes, from 1 minute to 30 minutes, from 5 minutes to 15 minutes, in particular for 10 minutes, in the processes as described herein.

The terms “reducing the incidence” as used herein mean that virus suspensions, viral seeds and/or preparations produced comprise (i) less adventitious agent, i.e. DNA, RNA and/or infectious adventitious particles, than if they were prepared using a method wherein the inoculum is not removed and washed off or the contact time is greater than that of the processes and methods of the invention, in particular greater than 120 minutes, or (ii) less adventitious agent, i.e. DNA, RNA and/or infectious adventitious particles, than initially present in the viral seed or virus preparation used to inoculate and infect cells.

The terms “removing adventitious agents” as used herein mean that virus suspensions, viral seeds and/or preparations produced according to the methods of the invention are free, or substantially free, of one or more adventitious agents that were present in the viral inoculum. The terms “substantially free” as used herein mean that there is less than 10⁶, 10⁵, 10⁴, 10³ 10², 10¹ or no detectable adventitious agents (i.e. DNA, RNA and/or infectious adventitious particles) per millilitre (ml). In particular, the preparations of the virus of interest, for example Rotavirus, obtainable according to the methods of the invention comprise less than 10⁴, suitably less than 10³, more suitably less than 10², or even less than 10¹ DNA copies/ml of adventitious agent, such as for instance PCV-1. The terms “removing adventitious agents” may also mean that virus suspensions produced according to processes of the invention comprise at least 90% less, suitably at least 95% less, more suitably, at least 99% less, and even at least 99.9% less of adventitious agent than the amount initially present in the virus seed or virus of interest preparation used to inoculate and infect cells.

The term “washing” as used herein means removing a solution, or substantially all of it, whether culture medium, such as for instance before infecting cells, or virus-containing solutions, such as the virus of interest inoculum used to infect cells, and replacing the solution with medium free of the virus, such as culture medium; virus-free medium may be substantially removed and replaced with fresh virus-free medium 1, 2, 3, 4, 5 or more times, as appropriate. Accordingly, in some embodiments of the invention, after removing the virus solution, cells are washed at least once, suitably twice, before further incubation to produce a population of replicated virus of interest.

A “virus seed” in its broadest meaning is to be understood as any virus solution or virus suspension or virus preparation used to infect cells by inoculating them, so as to replicate and propagate said virus on cells. A virus seed can also be called a virus inoculum, which inoculum is used for inoculating cells. For example, when considering vaccine manufacturing, the terms “virus seed” may be understood as a viral preparation from which all subsequent viruses for use in a vaccine and/or gene therapy are produced. The origin of the virus seed is known and the number of passages the virus has undergone is defined. For the purposes of the invention, the terms “virus seed” encompass Research seed, Master seed and Working seed and thus the present invention is suitable for producing any virus seed. Master seeds are homogenous virus preparations of known passage number derived from an original seed. Research seeds are any virus preparations derived from the original passaged 1, 2, 3, 4, 5, 6, or more times less than the Master seed. Working seeds are virus preparations derived from the Master seed, typically by 1 passage, but may have been passaged 2, 3, 4, 5 or more times than the Master seed. Viral seeds are generally stored frozen, for example at about −40° C. to about −70° C. and −196° C. in sterile polyethylene vials until used. The method according to the present invention is applicable to the production of any type of virus preparation, including any type of virus seeds.

The term “infection” is well known in the art. But for sake of clarity, “infection” encompasses at least the steps of adsorption, or attachment, of the virus onto the cells, entry, or penetration, of the virus into the cells, and possibly replication of the virus and release of newly formed viral particles able to further infect cells.

The terms “adventitious agents” as used herein mean any pathogen which is extraneous to a desired product, such as, for example, a virus for inclusion in a vaccine. The adventitious agents as described herein can be detected by any means known to the person skilled in the art. Methods available to the skilled person include but are not limited to immunological assays (e.g. Enzyme linked immunosorbant assay [ELISA] and Western blots), nucleic acid methods (e.g. polymerase chain reaction [PCR], Quantitative PCR [Q-PCR] Southern blot, Reverse transcriptase-PCR [RT-PCR], RT-Q-PCR etc.) and/or titration and or infectivity assays. Methods of detecting many adventitious agents are disclosed in WO2006/027698 (US2009081252A1). Methods of detecting adventitious viruses found in vaccines including porcine circovirus (PCV), simian retrovirus (SRV), avian leukosisvirus (ALV) and endogenous avian virus (AEV) are disclosed in Victoria JG et al., 2010 (Journal of Virology 84(12): 6033-6040). Methods for detecting PCV are also disclosed in Ouardani Metal., 2000 (Journal of Clinical Microbiology 38(4):1707).

The method for removing and/or reducing the incidence of adventitious agents is particularly useful in the production of a virus/viral seed. In addition, the processes and methods of the invention may be employed routinely as a safeguard against any contamination with any unknown adventitious agent. Therefore, it is envisioned that the processes and methods of the invention may be employed even if there is no detectable adventitious agent or no known adventitious agent. Accordingly, there is provided a process for producing a virus (in particular a viral seed), comprising inoculating susceptible cells by contacting them with a viral suspension, characterised in that the contact time between a viral suspension and susceptible cells is less than 120 minutes, for example less than about 90 minutes, about 60 minutes, about 45 minutes, about 30 minutes, 25 minutes, about 20 minutes, about 15 minutes, about 10 minutes or less than about 5 minutes, about 1 minute, or less than 45 seconds, in particular from about 5 seconds to about 1 hour, from about 10 seconds to about 55 minutes, from about 15 seconds to about 50 minutes from about 20 seconds to about 45 minutes, from about 30 seconds to about 40 minutes, from about 45 seconds to about 35 minutes or about 1 minute to about 30 minutes, for example from 10 to 30 seconds, from 20 seconds to 1 minute, from 10 seconds to about 30 minutes.

In a further embodiment of the invention there is provided a process for producing a virus seed, comprising the steps of:
i) inoculating susceptible cells with a viral inoculum comprising the virus and incubating the inoculated cells;
ii) washing said inoculated cells less than 120 minutes after inoculation, for example less than about 90 minutes, about 60 minutes, about 45 minutes, about 30 minutes, 25 minutes, about 20 minutes, about 15 minutes, about 10 minutes, about 5 minutes, about 1 minute, or less than 45 seconds after inoculation, in particular from about 5 seconds to about 1 hour, from about 10 seconds to about 55 minutes, from about 15 seconds to about 50 minutes from about 20 seconds to about 45 minutes, from about 30 seconds to about 40 minutes, from about 45 seconds to about 35 minutes or about 1 minute to about 30 minutes, for example from 10 to 30 seconds, from 20 seconds to 1 minute, from 10 seconds to about 30 minutes.

The present invention is also useful in the production of viruses when it is desirable to reduce and/or eliminate one or more adventitious agent, for example in the production of viruses for use in a vaccine/immunogenic composition and/or gene therapy.

Accordingly, in a further embodiment of the invention there is provided a process for producing a virus for use in medicine (in particular in a vaccine and/or gene therapy), comprising inoculating susceptible cells by contacting them with a viral inoculum or a virus preparation, characterised in that the contact time between the virus inoculum or the virus preparation and susceptible cells is less than 120 minutes, for example less than about 90 minutes, about 60 minutes, about 45 minutes, about 30 minutes, 25 minutes, about 20 minutes, about 15 minutes, about 10 minutes, about 5 minutes or less than about 1 minute for example, about 1 to about 120 minutes, about 1 minute to 90 minutes, about 1 to about 90 minutes, about 1 to about 60 minutes, about 1 to about 45 minutes, about 1 to about 30 minutes, about 1 to about 25 minutes, about 1 to about 20 minutes, about 1 to about 15 minutes, about 1 to about 10 minutes, about 1 to about 5 minutes, for example 1, 2, 3, 4, 5, 6, 7, 8, 9, 10, 11, 12, 13, 14, 15, 16, 17, 18, 19 or 20 minutes.

In another embodiment of the invention, there is provided a process for the replication of a virus for use in medicine (in particular a vaccine and/or gene therapy), comprising the steps:
(i) inoculating susceptible cells with a viral inoculum comprising the virus and incubating the inoculated cells;
(ii) washing said inoculated cells less than 120 minutes after inoculation, for example less than about 90 minutes, about 60 minutes, about 45 minutes, about 30 minutes, 25 minutes, about 20 minutes, about 15 minutes, about 10 minutes or less than about 5 minutes about 1 minute, or less than 45 seconds after inoculation, in particular from about 5 seconds to about 1 hour, from about 10 seconds to about 55 minutes, from about 15 seconds to about 50 minutes from about 20 seconds to about 45 minutes, from about 30 seconds to about 40 minutes, from about 45 seconds to about 35 minutes or about 1 minute to about 30 minutes, for example from 10 to 30 seconds, from 20 seconds to 1 minute, from 10 seconds to about 30 minutes.

Adventitious agent contamination can come from a variety of sources, for example from the cells in which the viruses replicate and are produced, from the viral inoculum used to infect cells (i.e. the source of the virus was contaminated or has subsequently become contaminated) or from media/reagents used during cell culture and/or virus replication. In one embodiment, there is provided processes and methods of the invention wherein the virus inoculum or virus of interest solution comprises one or more adventitious agents.

One purpose of the processes and methods of the invention is to reduce and/or eliminate the incidence/presence of adventitious agents. The term “adventitious agent” is well known in the art and as used herein means any extraneous contaminating infectious agent, such as virus other than the virus of interest which is intended to be produced, for example, for use in a vaccine and/or gene therapy. Accordingly, for the purpose of the invention, the virus intended to be produced by a method disclosed therein is called “virus of interest”, while the undesired contaminating adventitious viruses intended to be removed or reduced by the methods of the invention are referred to as “adventitious virus”. For example, whereas in one embodiment influenza virus is the virus of interest intended to be replicated, in an alternative embodiment influenza virus may be an adventitious virus if the virus of interest intended to be produced is a different virus, e.g. measles virus. Accordingly, the invention provides processes and methods as defined herein wherein the adventitious agent is an adventitious virus. The adventitious virus may be any virus described herein.

In a particular embodiment of the invention the adventitious virus is a non-enveloped virus such as Picornaviridae, Reoviridae, Birnaviridae (e.g. gumboro virus), Parvoviridae, Circoviridiae, Adenoviridae (e.g. adenoviruses, such as human or simian adenoviruses) or Polyoma viridae.

Other adventitious viruses include Herpesviridae (e.g. Herpes simplex 1 and Herpes simplex 2), Parainfluenza viruses (PIV) e.g. PIV-1, PIV-2 and PIV-3, Coronaviridae such as SARS coronavirus, Enteroviruses of the Picornaviridae family (e.g. Coxsackie viruses, or echoviruses), Rhinoviruses, Pneumovirinae, Morbilliviruses or Paramyxoviridae.

In one embodiment of the invention, the adventitious virus is a virus that does not replicate and/or cause disease in primates, for example humans. In a particular embodiment of the invention, the adventitious agent is porcine circovirus (PCV), and in further embodiments of the invention the adventitious agent is the porcine circovirus of Type 1 (PCV-1) and/or of type 2 PCV (PCV-2). Porcine circovirus (PCV) is a single stranded DNA virus (class II), that is non-enveloped with an un-segmented circular genome and is a member of the virus family Circoviridae. PCV-2 is believed to cause post-weaning multisystemic wasting syndrome in young piglets, marked by diarrhoea and an inability to gain weight. PCV-1 is related to PCV-2 but does not appear to cause disease in pigs. PCV-1 does not multiply in humans and is not known to cause illness in humans.

PCV-1 and/or PCV-2 are known as adventitious agents in HRV (human rotavirus) vaccines. Accordingly, in a particular embodiment, the virus of interest to be produced by processes and methods of the present invention, in particular, for use in a vaccine, is HRV and the adventitious agent is PCV, in particular PCV-1 and/or PCV-2.

Adventitious agents may be able to replicate in the cells or in culture medium. In a particular embodiment of the invention, the cells to be infected with the virus of interest are susceptible to one or more adventitious agents, whether the adventitious agent is derived from the cells, from the virus suspension or from reagents. The present invention is particularly useful where the cells are susceptible to the adventitious agents i.e. the adventitious agent(s) is/are able to replicate in the cells. Accordingly, there is provided a process or method wherein the cells are susceptible to infection with one or more of said adventitious agents.

The term “cells” as used herein means any cells that are used for the production of a virus of interest for use in a vaccine and/or gene therapy i.e. cells that are maintained in artificial conditions, in particular in vitro. The types of cells used will depend on the virus of interest to be produced because the cells must be susceptible to the virus to be produced, i.e. the virus of interest must be able to replicate in the cells. The skilled person knows which cell types are susceptible to a particular virus. A number of cell types are used to produce viruses that are suitable for use in vaccines and these include mammalian cells, such as MRC-5, Vero, CHO, FRHL-2, MDCK, PER.C6 or avian cells, such as Chicken Embryo Fibroblasts [CEF] or EBx® cells. In one embodiment of the invention, there is provided a process of the invention wherein the cells are selected from any cell type used to produce viral vaccines. In a further embodiment, the processes of the invention use cells selected from the group: MRC-5, Vero, CHO, FRHL-2, MDCK, PER.C6, EBx® cells and Chicken Embryo Fibroblasts [CEF]. In a particular embodiment of the invention the cells are Vero cells. Vero cells are susceptible, in particular, to HRV and Poliomyelitis virus. In one embodiment, the processes of the invention use Vero cells to produce HRV. Alternatively, Vero cells are used to produce Poliomyelitis virus. In another particular embodiment of the invention the cells are chicken cells or cell line EB14™ or duck cells or cell line EB24™ or EB66™, such as manufactured in, or as otherwise disclosed in, WO2008129058 or WO2003076601 (produced by Vivalis: see also www.vivalis.com). MDCK cells, PER.C6 and EB66^{TM} are known to be susceptible, in particular, to infection with Influenza virus. CEF cells are, in particular, susceptible to infection with Measles and Mumps virus.

The cells of the invention are typically grown in vitro. The cells may be adherent cells such as in the form of a monolayer i.e. attached to the surface of a container or attached to a support in suspension in the culture medium, such as attached to microcarriers. Preferred microcarriers are manufactured from synthetic materials including cross-linked dextran, such as Cytodex® I which is preferred. In another embodiment, the cells may be in suspension in the culture medium.

In a particular embodiment of the invention, the cells used in the processes and methods of the invention are mammalian, avian or insect cells. In a specific embodiment, the cells used in the processes and methods of the invention are derived from a mammal (i.e. they are mammalian). In a particular embodiment, the cells used in the processes and methods described herein are derived from a primate, such as an African Green monkey.

The processes and methods of the invention are used, in particular, to produce viruses used in the manufacture of vaccines. Accordingly, in one embodiment, there are provided processes and methods of the invention wherein the virus of interest is a virus that infects and/or causes disease in primates, in particular humans. Viruses produced by, and thus used in the processes or methods of the invention may be attenuated, i.e. the virus is viable (i.e. alive) but is either less virulent compared to the wild type strain or avirulent. In one embodiment, there is provided processes and methods of invention, wherein the virus of interest is an attenuated virus.

In an alternative embodiment, the virus is not attenuated. In these circumstances, following incubation of infected cells (i.e. replication and propagation of the virus) the non-attenuated virus is subsequently inactivated (i.e. killed) if the virus causes disease in the host to which the vaccine is administered, for example poliomyelitis virus. In some circumstances, live viruses do not need to be inactivated as the live virus does not cause disease in humans yet elicits an immune response against another organism. For example, the immune response raised protects against a related virus and a pathogen or in an alternative embodiment, the virus may encode one or more antigens derived from a different pathogen which elicits a protective immune response against said pathogen.

In one embodiment, there is provided processes and methods of the invention wherein the virus of interest is selected from the group: measles, mumps, rubella, human papilloma virus (HPV), Baculovirus, influenza, varicella zoster virus (VZV), poliomyelitis virus, Epstein bar virus (EBV), Human Immunodeficiency virus (HIV), Herpes simplex virus (HSV), Hepatitis B (HBV), Hepatitis C (HCV), Hepatitis E (HBV), Dengue virus, cytomegalovirus (CMV) respiratory syncytial virus (RSV), rabies virus, human rotavirus (HRV), Adenovirus or Hepatitis A (HAV).

In a particular embodiment of the invention, the virus is human Rotavirus (HRV). Human rotavirus causes severe diarrhoea in infant and young children and is a double stranded RNA virus from the family Reoviridae. There are five species of Rotavirus, A, B, C, D and E of which A is the most common to infect humans.

HRV species A can be characterised by serotypes. The glycoprotein VP7 defines G-types and the protease-sensitive protein VP4 defines P-types. Strains are generally designated by their G serotype specificities (e.g. serotypes G1 to G4 and G9), and the P-type is indicated by a number and a letter for the P-serotype and by a number in square brackets for the corresponding P-genotype. In a particular embodiment of the invention, the Rotavirus is selected is a HRV A. In a further embodiment, the HRV is selected from the group comprising serotypes G1, G2, G3, G4, G9, P1 or P8. In a further embodiment, the virus of interest produced by methods of the present invention is a human Rotavirus of a G1P[8] type. The processes of the invention are also applicable to the production of reassortant Rotaviruses, including for example human, bovine, and human/bovine reassortants. Accordingly, the present invention contemplates using a solution comprising any type of reassortant Rotavirus to infect cells according to processes as disclosed therein.

The multiplicity of infection (MOI) will depend on the virus of interest, as well as any adventitious agents in the virus inoculum or virus solution. The skilled person fully understands the term “MOI”, but for clarity MOI is the ratio of viruses to cells at infection. Increasing the number of MOI of the virus of interest when infecting susceptible cells may result in an improved yield of the virus of interest. However, increasing this number may also lead to a concomitant increase of the production of the contaminating adventitious agent. It is within the skilled person's capacity to make the MOI number vary, and determine which number(s) give(s) an appropriate yield of the virus of interest, while maintaining the level of the contaminating adventitious agent at an acceptable level. Depending on the application intended for the virus of interest produced according to the processes of the invention, the minimal and maximal threshold of contaminating adventitious agents can change. Accordingly, the MOI number will be adapted to the desired yield of virus of interest and to the desired level of contaminating adventitious agents. In one embodiment, there is provided processes and methods of the invention wherein the cells are suitably infected by contacting them with the virus of interest at a multiplicity of infection (MOI) of about 1.0, about 0.5, about 0.1, about 0.01, about 0.001, about 0.0001 or about 0.00001. In a particular embodiment, the cells, in particular Vero cells, are contacted with a virus, such as for instance HRV, possibly contaminated with PCV-1, at a MOI of 0.1.

The temperature conditions for virus infection may vary. Temperature may range from 32° C. to 39° C. depending on the virus type. For Influenza virus production, cell culture infection may vary depending on the strain which is produced. Influenza virus infection is suitably performed at a temperature ranging from 32° C. to 35° C., suitably at 33° C. Alternatively, Rotavirus infection is suitably performed at 37° C. Proteases, typically trypsin, may be added to the cell culture depending on the virus strain, to optimize viral replication and propagation. For example, Influenza virus and Rotavirus production can be improved by using trypsin in cell culture. The protease can be added at any suitable stage during the culture. It can be added in the solution comprising the virus of interest to be contacted with susceptible cells and/or to the culture medium used to incubate cells after they were contacted with the virus. Tryspin is suitably of non-animal origin, that is to say the protease is not purified from an animal source. It is suitably recombinantly produced in a micro-organism, such as bacterial, yeast or plant. Suitable examples of recombinant trypsin are Trypzean, a recombinant trypsin produced in corn (Prodigen, 101 Gateway Blvd, Suite 100 College Station, Tex. 77845. Manufacturer code : TRY), or TrpLE (Invitrogen) which is a trypsin-like enzyme expressed in fungus (WO2004/020612). Alternatively, the trypsin can be of animal origin, suitably of porcine origin. As a non-limiting example, a suitable concentration of tryspin to be added according to the above, ranges from 5 μg/ml to 25 μg/ml, suitably 7.5 μg/ml or 15 μg/ml.

In a further embodiment of the invention, there is provided a process or method as defined herein further comprising the step of incubating the cells which have been contacted with a virus to produce a population of replicated virus of interest. The contacted cells are incubated under conditions that are suitable for viral replication and production. The temperature may vary according to the virus to be produced. Accordingly, in one embodiment of the invention there is provided a process or method of the invention wherein the cells which have been contacted with a solution comprising a virus of interest are incubated at a temperature higher than 25° C., suitably lower than 41° C., such as for example between 25° C. and 41° C., for example between 34° C. and 40° C., 35° C. and 39° C., 36° C. and 38° C., for example about 37° C±1° C.

In a still further embodiment of the invention, there is provided a process or method as defined herein further comprising the step of collecting the population of replicated virus of interest. Once infected, cells may release into the culture medium newly replicated and formed virus particles, due to spontaneous lysis of host cells, also called passive lysis, or due to budding phenomenon. Accordingly, one possible way of collecting the produced virus of interest is to collect the virus-containing culture medium. Alternatively, after virus infection, the produced virus of interest may be harvested by employing external factor to lyse cells, also called active lysis. Methods that can be used for active cell lysis are known. Useful methods in this respect are for example, freeze-thaw, solid shear, hypertonic and/or hypotonic lysis, liquid shear, high pressure extrusion, detergent lysis, or any combination thereof. Collecting the virus-containing culture medium and lysing cells may also be combined to harvest the virus of interest produced according to the processes of the invention.

The duration of incubation before collecting the virus is dependent on the virus of interest to be produced. For example, the duration of incubation may be from about 1 to about 30 days. In a particular embodiment, there is provided a process or method of the invention wherein the cells which have been contacted with a virus of interest are incubated for about 2 to 10 days, for example 3 to 9 days, 4 to 8 days, 5 to 7 days, or 5 to 6 days. The optimal time to harvest the produced virus is usually based on the determination of the infection peak. For example, when the virus of interest is cytopathic, i.e. provokes spontaneous lysis of cells after infection, the CPE (CytoPathic Effect) can be measured by monitoring the morphological changes occurring in cells after virus inoculation, including cell rounding, disorientation, swelling or shrinking, death, detachment from the surface. The detection of a specific viral antigen may also be monitored by standard techniques of protein detection, such as a Western-blot analysis. Harvest can then be collected when the desired detection level is achieved. The content of an antigen may also be monitored any time post-inoculation of the cells with the virus, by the SRD assay (Wood, J M, et al. (1977). J. Biol. Standard. 5, 237-247), which is a technique familiar to a person skilled in the art. This technique is particularly suitable to determine the HA content of Influenza virus. Alternatively, the production or yield of a virus of interest can be analysed by measuring the Cell Culture Infectious dose (CCID₅₀/ml), which represents the amount of a virus capable of infecting 50% of cells. A series of successive dilutions of the infectious virus samples to be tested are performed and part of each dilution is used for inoculating susceptible cells. After incubating the cells for a few days, so that the virus can replicate, the presence of the virus may be detected by two reading methods known to the skilled person, the analysis of the cytopathic effect (CPE) in cells and/or the hemagglutination assay with chicken red blood cells performed on the culture supernatant. The viral titer is then calculated according to the Reed and Muench method (Reed, L. J. and Muench, H., 1938, The American Journal of Hygiene 27: 493-497). In one embodiment, there is provided a process of the invention wherein the cells which have been contacted with a virus of interest are incubated for 6 days or 7 days, and then the virus is possibly harvested by collecting the cell culture medium.

Depending on the process, following incubation, the virus suspension may be used as a viral seed (research, master seed or working seed) or directly used to produce a vaccine. If the virus suspension is to be used at a later date the virus may be frozen under any conditions that maintain viability of the virus. In particular, the virus may be stored at a temperature between about −(minus) 40° C. and about −70° C., for example about −45° C., about −60 ° C., about −70° C. or about −196° C. Accordingly, in one embodiment, there is provided a process or method of the invention further comprising the step of freezing the virus suspension.

Further to its production on cell culture, the virus of interest can be optionally purified. One or more purification steps, know in the art, can be implemented during the processes of the invention. Accordingly, in some embodiments of the invention, the processes as described therein comprise at least one step selected from clarification, ultrafiltration/diafiltration, ultracentrifugation, such as density, in particular, sucrose gradient density ultracentrifugation and chromatography, or any combination thereof. Depending on the purity level that is desired, the above steps may be combined in any way. In a particular embodiment, there is provided processes of the invention further comprising the step of clarifying the population of replicated virus of interest or the virus supension produced on cell culture to remove cell debris. The term “clarify” as used herein means separating the virus from cellular material, such as intact floating cells and/or cell debris. The virus suspension may be clarified by any means known to the skilled person and include, but are not limited to filtration (e.g. with a 0.5 μm filter membrane) or microfiltration. A suitable alternative clarification means is centrifugation. In particular, the virus suspension obtained according to the process of the invention is centrifuged at about 1000 rpm for a few minutes, such as for example 10 minutes.

The virus suspension of the invention may be frozen before it is clarified and thus in one embodiment, there is provided a process or method as defined herein where the virus suspension is frozen. The virus may be frozen under any conditions that maintain viability of the virus. In particular, the virus may be stored at a temperature between about −(minus) 40° C. and about −70° C., for example about −45° C., about −60° C. or about −70° C.

If the virus is frozen prior to clarification, the virus suspension will need to be thawed/defrosted prior to clarification. In one embodiment, there is provided processes and methods of the invention further comprising the step of thawing the frozen virus suspension and then clarifying the virus suspension to remove cell debris.

In order to remove the DNA derived from cells, the virus suspension may be treated with a nuclease know to the skilled person including, but not limited to, Benzonase™. Accordingly, there is provided processes and methods of the invention wherein virus suspension is treated to remove DNA derived from the cells, for example using Benzonase™.

The virus suspension or population of replicated virus of interest produced by processes or methods of the invention may require concentrating, i.e. the volume of liquid in which the virus is suspended may need to be reduced, e.g. for formulation into a suitable volume for vaccination or so that the cells can be resuspended in an appropriate volume for inoculation. Accordingly, in a further embodiment, the virus suspension produced by processes and methods of the invention may be concentrated by means known to the skilled person which include but are not limited to ultrafiltration.

In embodiments of the invention wherein the virus is not attenuated and causes disease in the host, it is necessary to inactivate the virus so that the virus elicits a protective immune response yet does not cause disease in the host to which the virus has been administered. Accordingly, the invention provides processes of the invention comprising the step of inactivating the virus. Methods of inactivation are known to the skilled person.

In a particular embodiment of the invention, the virus (e.g. influenza virus) is inactivated by “splitting” i.e. disrupting the virus to produce viral fragments using detergents. Alternatively, or in addition, the virus of interest obtained according to the processes of the invention can be inactivated by chemical treatments, such as using beta-propiolactone (BPL) or formaldehyde, and physical treatments, such as UV irradiation, or a combination or both.

In order to remove larger adventitious agents such as bacteria from the virus suspensions as defined herein, the virus suspension may be sterile filtered. Accordingly, in a further embodiment, there is provided processes and methods as defined herein further comprising the step of sterile filtering the virus suspension through a sterile grade filter membrane. Suitable means for sterile filtering are well known to the skilled person.

Sterile filtration is performed using sterile grade filter membranes. A sterile grade filter membrane is a filter membrane that produces a sterile effluent after being challenged by microorganisms at a challenge level of greater than or equal to 1×10⁷/cm2 of effective filtration area. Sterile grade filters are well known to the person skilled in the art of the invention and have a pore size of about 0.2 μm, and thus include filters with a pore size of about 0.22 μm.

In a further embodiment of the invention, the virus suspension produced by the processes or methods as defined herein further comprising the step of filtering the virus suspension through one or more filters (e.g. a sterile grade filter). In a particular embodiment of the invention, there is provided a process or methods as defined herein wherein the virus suspension is filtered through a filter with a pore size of about 0.5 μm (for example 0.4 μm to 0.6 μm) and subsequently through a sterile grade filter (for example about 0.2 μm or 0.22 μm).

Any of the steps of the processes of the invention may be repeated, for example 1, 2, 3, 4, 5, 6 or more times. In the sense of the present invention, the series of successive steps a) to c) of the processes of the invention is also called “virus passaging”. For example, in a particular embodiment, there is provided the processes of the invention wherein the virus is passaged 1, 2, 3, 4, 5 or more times. In a particular embodiment of the invention, the virus is further passaged either once or twice in addition to the first series of steps a) to c). Accordingly, in a particular embodiment there is provided processes of the invention wherein the series of steps a) to c) is repeated at least once, using the population of replicated virus of interest obtained at step c) as the solution comprising the virus of interest for contacting the population of cells according to further step a). Suitably, the series of steps a) to c) is repeated more than once, for example 1, 2, 3, 4, 5, 6 or more times. Passaging the virus more than once, such as twice or three times, by repeating the steps a) to c) of the processes of the invention allows, in particular, to further reduce the presence of contaminating adventitious agents.

The population of replicated virus of interest produced by the processes of the invention, or passaged once, can be optionally stored (for example frozen at −70° C.), before being subject to a further virus passage. In a particular embodiment, the virus is passaged a second time wherein the virus suspension produced following the first passage is optionally stored (for example frozen at −70° C.) before being used to infect new cells.

The processes of the invention result in virus suspensions or virus preparations, such as viral seeds, with a reduced incidence or reduced presence and/or absence of one or more adventitious agents. Accordingly, in a particular embodiment of the invention there is provided a virus preparation, such as a viral seed, obtainable by any of the processes for producing a virus in accordance with the present invention. In a further embodiment, there is provided an immunogenic composition, such as a vaccine, produced and/or derived from the virus preparation, such a viral seed, obtainable by any of the processes of the invention.

Vaccines or immunogenic compositions made by or derived from virus preparations, such as viral seeds, produced by the processes of the invention may comprise one or more viruses. In a particular embodiment of the invention, there is provided a vaccine produced and/or derived from the virus suspension obtainable by any of the processes for producing a virus, wherein the virus is HRV.

In a particular embodiment of the invention there is provided an immunogenic composition, such as a vaccine, comprising a virus, wherein the composition or the vaccine is substantially free of an adventitious agent. In a particular embodiment, the composition or the vaccine substantially free of an adventitious agent comprises HRV. In an even more particular embodiment, there is provided an immunogenic composition or a vaccine comprising HRV, wherein said composition or said vaccine is substantially free of PCV-1 and/or PCV-2.

Similarly, the processes of the invention may be used for producing viruses for use in vaccination and/or gene therapy and thus accordingly, the present invention also provides a virus and/or a virus suspension obtainable by any of the processes or methods as defined herein.

Vaccines or immunogenic compositions may comprise one or more viruses for use in a vaccine produced by processes of the invention. In a particular embodiment of the invention, there is provided a vaccine comprising a virus of interest produced by the processes of the invention wherein the virus is HRV.

HRV vaccines of the invention may comprise more than 1 serotype of HRV and in a particular embodiment of the invention the HRV vaccine comprises 5 or more HRV serotypes (in particular G1, G2, G3, G4, G9, P1 or P8). The Rotaviruses or antigens thereof can be reassortants, such as human, bovine or human/bovine reassortants. Alternatively, the Rotavirus vaccines of the invention comprise a G1 P[8] strain, optionally attenuated.

In a further embodiment of the invention, there is provided an immunogenic composition or a vaccine comprising a virus free, or substantially free, of an adventitious agent. In another embodiment there is provided a HRV vaccine as defined herein free, or substantially free, of an adventitious agent. In a particular embodiment of the invention, there is provided an HRV vaccine as defined herein substantially free of PCV-1 and/or PCV-2.

In a further embodiment of the invention, there is provided a vaccine comprising one or more of the viruses as defined herein. In a particular embodiment of the invention, the vaccine comprises measles, mumps, rubella, varicella zoster virus (VZV) or any combination thereof. In another particular embodiment the vaccine comprises influenza virus.

In immunogenic compositions or vaccines of the invention comprising one or more viruses, one, some (for example, 2, 3 or 4) or all of the viruses may be produced by the processes or methods of the invention; some may be produced by other methods. It is envisioned that each virus is produced separately and combined in the final vaccine formulation.

In a further embodiment, there is provided a vaccine comprising one or more of the viruses of interest (for example inactivated Poliomyelitis Virus) and one or more antigens derived from one or more bacteria, for example, diphtheria toxoid, tetanus toxoid, pertussis toxoid, a Haemophilus influenzae polysaccharide or an antigen derived from Neisseria (e.g. N. meningitidis) or combination thereof.

In particular embodiments, the vaccines or immunogenic compositions of the invention do not comprise a mercurial material (e.g. thiomersal) and/or animal derived products (e.g. serum, in particular bovine derived serum).

Vaccines and or immunogenic compositions of the invention as described herein may further comprise one or more adjuvants such as alum (aluminium hydroxide, aluminium phosphate or a combination thereof), a Toll-like receptor ligand (e.g. a TLR 4 ligand such as 3D-MPL or a TLR 9 ligand such as CpG oligonucleotide), a saponin (e.g. QS21), liposomes, an oil in water emulsion (for example AS03 or MF59), or a combination thereof.

Vaccines and immunogenic compositions of the present invention are suitable for use in medicine and in particular for use in the prevention and/or treatment of disease in a mammal (in particular, humans). Accordingly, in one embodiment, there is provided vaccines and immunogenic compositions of the invention as described herein for use in medicine. In a further embodiment, there is provided vaccines and immunogenic compositions of the invention as described herein for use in the prophylaxis and/or treatment against a disease or condition. In particular, when the vaccines comprise Rotaviruses, said vaccines are used to prevent and/or treat rotavirus-associated gastro-enteritis.

In a further embodiment there is provided the use of vaccines and immunogenic compositions of the invention as described herein in the manufacture of a medicament for the prophylaxis and/or treatment against a disease or condition. In another embodiment there is provided a method of treatment comprising the step of administering a vaccine and immunogenic composition of the invention as described herein.

Embodiments herein relating to “vaccine compositions” or “vaccines” of the invention are also applicable to embodiments relating to “immunogenic compositions” of the invention, and vice versa.

The terms “comprising”, “comprise” and “comprises” herein are intended by the inventors to be optionally substitutable with the terms “consisting of”, “consist of” and “consists of”, respectively, in every instance.

The term “about” in relation to a numerical value x means x±5% or 10%.

The word “substantially” does not exclude “completely” and may be omitted from the definition of the invention.

The invention will now be described further by way of reference to the following, non-limiting examples.

### EXAMPLE 1

### Overview of Protocols

Bulk production of Rotavirus was performed by growing a Vero cell bank through different pre-culture phases. The adventitious agent, PCV-1, was present in the Rotavirus seed used to infect the cells, as DNA, RNA and viral particles including infectious particles. After preparation of the required cell culture supports, the virus was inoculated for multiplication and production by contacting the cells with a rotavirus suspension and cells were left incubated with the virus for 5 to 7 days. The produced virus was harvested after the desired incubation time by collecting the supernatant. The harvested virus was frozen and stored at −70° C.

### 1.1 Cell Culture Preparation

A Vero cell culture was prepared in serum-free conditions using the VP-SFM culture medium (Invitrogen, No. 11681420) as growth medium. T-175 flasks (175 cm²) or T-25 flasks (25 cm²) were seeded with 50,000 to 100,000 cells/cm² and grew for 3 to 7 days.

### 1.2 Virus Preparation

The required amount of Rotavirus inoculum (originating from a human G1P[8] strain) was thawed at 37° C. in a water bath under agitation. The virus was then activated in solution in DMEM (Dulbecco's Modified Eagle Medium) supplemented with porcine trypsin (Sigma) at a final concentration of 7.5 to 20 μg/ml at room temperature for 30 minutes. After activation, the virus solution was diluted by addition of fresh DMEM supplemented with trypsin at a final concentration of 7.5 μg/ml in order to obtain the virus concentration required to target a MOI of 10⁻¹, 10⁻³, 10⁻⁴ or 10⁻⁵.

### 1.3 Virus Infection

Growth medium was discarded from the T-flasks and the cell layers were washed with 50 ml of DMEM. The cells were then infected with Rotavirus as follows: they were contacted with the desired MOI number of the virus by replacing the washing medium with 45 ml of the above diluted and activated virus solution. The cells were then left contacted with the virus solution at 37° C. for a time less than or equal to 1 minute or for 30 minutes.

### 1.4 Virus Production

After the indicated contact time, the virus solution was removed. The cell layers were washed 2 times with DMEM. The T-flasks were then further incubated at 37° C. for 5 to 7 days in DMEM supplemented with 15 μg/ml of trypsin. As a control, some flasks were not washed after contacting the cells with the virus solution (i.e. the virus solution was not removed), but left incubated with the virus solution at 37° C. for 5 to 7 days. Virus-containing supernatant was collected at day 5 to 7 post-infection. The material was stored at −70° C.

### 1.5 Method for Measuring Rotavirus Titer—Virus Titration in ffu/ml

MA-104 cells were cultured in DMEM supplemented with 10% foetal bovine serum. 96 well plates were seeded with 20,000 cells per well. Cells were left incubated at 37° C. for 4 days. Before adding samples of virus-containing supernatant to be titrated to the cells, cells were gently washed three times with DMEM. Then, 120 pl of titration medium, i.e DMEM supplemented with 8 μg/ml of trypsin was added to the cells. A first 2-fold dilution of the sample of virus-containing supernatant was prepared and the diluted virus was activated for 30 minutes at room temperature. The activated diluted virus was used to prepare further serial 2-fold dilutions performed directly into the 96-well plate comprising the MA-104 cells as prepared above. 120 μl of the activated diluted virus was added to a first series of wells, each containing 120 μl of titration medium. Then, 120 μl of those further diluted virus were added to a next series of wells, each containing 120 μl of titration medium, etc . . . Plates were centrifuged at 2000 rpm for 1 h30 at 30° C. 96-well plates were then incubated with the dilutions of virus-containing samples for 16 h. Then, the cells were washed once with DMEM and fixed by incubating them in cold acetone 80% (−20° C.) for 15 min to 25 min at −20° C. Acetone solution was then removed, and plates were dried. A solution comprising a monoclonal antibody directed to VP6 protein was then added to cells in order to detect and quantitate the presence of Rotavirus in the samples and incubated for 1 hour at 37° C. Said presence was revealed by immunofluoresence using a secondary anti-mouse antibody coupled to FITC incubated on cells for 1 hour at 37° C. One cell positive for fluorescence (called focus) indicates that said cell has been infected. It is estimated that a 16 h incubation of cells with a population of viruses allows cells to be infected with one virus only. Accordingly, the titer of a virus solution in ffu/ml corresponds to the number of foci detected in a well corrected by the dilution factor. ffu is for foci-forming unit.

### 1.6 Method for Measuring Rotavirus Titer—Virus Titration in Log CCID50/ml

Virus inactivation was assessed by measuring the viral titration through the TCID₅₀ assay (Tissue-Culture-Infectious-Dose). At the end of the overnight incubation, a sample from every BPL conditions within each experiment was collected to test its infectiousness in order to evaluate the efficacy of BPL inactivation. A series of successive dilutions of the samples to be tested are performed. 50 μl of each dilution is inoculated in 10 replicates into a 96 wells microplate containing MDCK cells, 8 dilutions being inoculated for each sample to be tested. Plates are then incubated for 5-7 days at 35° C., so that the virus, if infectious, can replicate in cells. The presence of infectious virus in cells is detected by monitoring the Cytopathic effect (CPE) on the cells by microscopy. A suspension of infectious virus is used as a positive control to demonstrate cellular susceptibility and non-inoculated cultures are used as negative control. The number of wells where CPE is detected is scored for each dilution as infected cells and the viral titer is calculated according to Reed and Muench method (Reed, L. J. and Muench, H., 1938, The American Journal of Hygiene 27: 493-497).

### 1.7 Method for Measuring PCV-1 DNA Content—Q-PCR Assay (Quantitative PCR)

Quantitative PCR (Q-PCR) also known as real-time amplification assay is based on the use of a fluorescent probe to detect the accumulation of the amplified product after each PCR cycle. PCR product amplification is monitored in real-time via a fluorogenic probe that binds specifically to the amplified product. As long as the probe is not bound, no fluorescence is emitted. Q-PCR assays were performed as follows. Test samples single-stranded DNA was extracted using a commercially available system (QlAamp Viral RNA) following the manufacturer's instructions. Extracted PCV-1 DNA was amplified by Q-PCR using specific primers and probe (see Table 1). The cycling conditions used for the Q-PCR assays are detailed in Table 2. The assay was run in 96-wells microplates using an ABI PRISM 7900 HT apparatus from Applied Biosystem. Dedicated rooms and material were assigned to each step (PCR pre-mix preparation, DNA extraction, PCR assembly, PCR amplification) in order to avoid contamination of the samples.

Numerous positive and negative controls were used to validate the assay:
Extraction where no DNA was present,
PCR reaction where no was DNA present,
PCV-1 DNA of known concentration used as positive controls of the PCR reaction and for drawing a standard curve allowing quantitation of the samples to be tested (from 10 to 10⁷ copies),

Each sample was run in duplicate.

TABLE 1
Primers and probes used in the Q-PCR assays

| | | |
|---|---|---|
| | Location | |
| | of primer | |
| Primer | (nucle- | |
| name | otide) | Nucleotide sequence |

*CAP Q-PCR assay

| | | | |
|---|---|---|---|
| Primers | PCV1_TMF1 | 1599-1615 | 5′-CGGCGCCATCTGTAACG |
| | PCV1_TMR1 | 1640-1661 | 5′-ATATGGTCTTCTCCGGA |
| | | | GGATG |
| probe | PCV1_TMP1 | 71618-1638 | 5′-TTCTGAAGGCGGGGTGT |
| | | | GCCA |

*CAP is for capside

TABLE 2
Q-PCR reaction conditions

| | Initial Steps | | × 40 Cycles | |
|---|---|---|---|---|
| | Hold | Hold | Cycle | |
| Temperature | 50° C. | 95° C. | 95° C. | 60° C. |
| Time | 2 minutes | 10 minutes | 15 seconds | 1 minute |
| Step Description | UNG | Taq Activation | Denaturation | Anneal/ |
| | Activation | | | Extend |

UNG: Uracil-DNA glycosilase

### 1.8 PCV-1 Infectivity Assay

A cell culture based assay was performed in order to assess the presence of PCV-1 replication-competent particles in Rotavirus bulk, i.e. of PCV-1 infectious particles. The assay was run on PCV-1-free Vero cells. Rotavirus from the Rotavirus-containing supernatant samples resulting from the clearance trials, as described in the below Examples, was neutralized by adding a Rotavirus-specific monoclonal antibody. Sub-confluent Vero cells seeded in flasks the day before were contacted with the above neutralized samples. After a contact time of 2 hours at 37° C., the culture supernatant was removed, the cells were washed and fresh culture medium was added. As negative controls, Vero cells were not contacted with Rotavirus-containing samples and filled with culture medium. Positive controls were produced by the infection of Vero cells with 1×10³ CCID₅₀ from a PCV-1 viral stock in culture medium with or without Rotavirus-specific monoclonal antibodies. The flasks were incubated at 37° C. with regular cells passages (twice a week). 14 days after inoculation with the virus samples, cells were collected and the presence of PCV-1 transcripts was tested by reverse transcriptase rep' RT-Q-PCR assay, as described below, which transcripts, if present reflect the ability of PCV-1 to replicate, and thus the presence of infectious PCV-1 particles.

2×10⁶ Vero cells were washed with PBS and the cell pellet was subject to RNA extraction using the High pure RNA kit (Roche) with DNAse treatment following the manufacturer's instructions. RNA was eluted in 50 μl elution buffer and treated with TurboDNAse. RT reactions were performed for each sample by using Q script cDNA synthesis (Quanta). Briefly, 5 μl of RNA was reverse transcribed in a volume of 20 μl and incubated for 5 min at 22° C., followed by 30 min at 42° C. and 5 min at 85° C. Taqman Q-PCR directed against the Rep' transcript was used, amplifying a 111 by fragment. The primers and probe used were those disclosed in Mankertz & Hillenbrand, 2001 (Virology 279: 429-438). The Q-PCR was performed by using the Gene expression master mix (ABI), in a final volume of 25 μl containing 2.5 μl of cDNA. After 2 min at 50° C. and 10 min at 95° C., 40 cycles were performed consisting of 15 sec at 95° C. and 1 min at 60° C.

### Example 2

### Effect of Reducing the Virus Contact Time with Cells

2.1 Three independent experiments (Run 1, Run 2 and Run 3) were initiated to assess the effect of removing the virus solution after a reduced contact time, as compared with the control where the virus solution was not removed but maintained until the virus harvest time. The virus MOI was 10⁻¹ and the conditions of cell culture and virus infection were identical and as described in Example 1. Virus-containing supernatants were harvested after 6 or 7 days.

In Run 1, PCV1-free Vero cell cultures were grown in VP-SFM in T-175 flasks, as described in section 1.1. They were washed with DMEM and then infected by contacting them with a Rotavirus solution (diluted in DMEM supplemented with 15 μg/ml of trypsin) at a MOI of 10⁻¹. After 10 to 30 seconds at 37° C., the virus solution was removed. The cell layer was washed two times with DMEM. Then, DMEM supplemented with 7.5 μg/ml of trypsin was added to the cells and the cells were further incubated at 37° C. for 6 days or 7 days. Prior to the contact, the virus was specifically activated as described in section 1.2 in DMEM supplemented with 15 μg/ml of trypsin. As control, the virus solution was not removed from some flasks and contact with the virus solution was maintained all along the incubation period (6 or 7 days). Virus-containing supernatants were harvested 6 days or 7 days post-infection and were frozen at −70° C.

Based on the same method, a second virus passage (VP2) was done in Vero cells prepared similarly to Run 1. A virus sample from the above-harvested supernatant of Run 1 was thawed and specifically activated as described in section 1.2 in DMEM supplemented with 15 μg/ml of trypsin. Cells were washed with DMEM and infected by contacting them with 10⁻¹ MOI of the activated virus solution (diluted in DMEM containing 7.5 μg/ml of trypsin). After a contact time of 10 to 30 seconds, the virus solution was removed and the cells were washed two times with DMEM and further incubated in DMEM supplemented with 7.5 μg/ml of trypsin at 37° C. for 6 days. Then, virus-containing supernatant was collected and stored at −70° C.

A third virus passage (VP3) was carried out in T-25 flasks with the same method. A virus sample from the above-collected supernatant of the second virus passage of Run 1 was thawed and specifically activated as described in section 1.2 in DMEM supplemented with 15 μg/ml of trypsin. Cells were washed with DMEM and infected by contacting them with 10⁻¹ MOI of the activated virus solution (diluted in DMEM containing 7.5 μg/ml of trypsin). After a contact time of 10 to 30 seconds, the virus solution was removed and cells were washed two times with DMEM, and further incubated in DMEM supplemented with 7.5 μg/ml of trypsin at 37° C. for 7 days. Then, virus-containing supernatant was harvested and stored at −70° C.

In Run 2, the virus passages were done either in T-175 flasks (VP1) or T-25 flasks (VP2) according to the above method. PCV-1-free Vero cell cultures were grown in VP-SFM, as described in section 1.1. The cells were washed with DMEM, and then infected by contacting them with 10⁻¹ of a rotavirus solution (diluted in DMEM medium supplemented with 15 μg/ml of trypsin). After either 10 to 30 seconds of contact (Run 2b) or 30 minutes of contact (Run 2a) at 37° C., the virus solution was removed. The cell layer was washed two times with DMEM. Then, DMEM supplemented with 7.5 μg/ml of trypsin was added to the cells and the cells were further incubated at 37° C. for 6 days. Then, virus-containing supernatant was harvested and stored at −70° C. A second virus passage (VP2) was done in Vero cells prepared similarly to the above. A virus sample from the above-harvested supernatant of Run 2b was thawed and specifically activated as described above. Cells were washed with DMEM and infected by contacting them with 10⁻¹ MOI of the activated virus solution (diluted in DMEM containing 7.5 μg/ml of trypsin). After a contact time of 10 to 30 seconds, the virus solution was removed and the cells were washed two times with DMEM and further incubated in DMEM supplemented with 7.5 μg/ml of trypsin at 37° C. for 7 days. Then, virus-containing supernatant was collected and stored at −70° C.

In Run 3, one virus passage (VP1) was performed in Cell Factory according to the above method. Prior to contacting the cells with the rotavirus solution, the virus was specifically activated as described in section 1.2 in DMEM supplemented with 20 μg/ml of trypsin.

Cells were washed with DMEM and infected by contacting them with 10⁻¹ MOI of the activated virus solution (diluted in DMEM medium supplemented with 15 μg/ml of trypsin). The contact time was close to 1 minute. The virus solution was then removed. Cells were washed two times with DMEM and further incubated at 37° C. for 6 days in DMEM supplemented with 7.5 μg/ml of trypsin. Then, the virus-containing supernatant was harvested.

All of the above Rotavirus-containing supernatants were subject to a titration assay allowing to measure Rotavirus titer and to a Q-PCR assay allowing to determine the PCV-1 DNA content, as described in the above sections 1.5 and 1.7, respectively. The results are presented in Table 3. They are expressed in log ffu/ml and DNA copies/ml, respectively.

TABLE 3
Effect of reducing virus contact time with the cells on Rotavirus titer and
PCV-1 DNA content

| | | | PCV-1 | Rotavirus |
|---|---|---|---|---|
| Virus passage | Virus | Harvest | (DNA | titer |
| (VP) | Contact time | day | copies/ml) | (log ffu*/ml) |
| VP1 | 6 days | D6 | 4.4 × 10⁶ | 6.8 |
| Control Run 1 | 7 days | D7 | Not done | 7.4 |
| VP1 | | | | |
| Run 1 | 10 to 30 sec | D6 | 1.7 × 10³ | 6.2 |
| | | D7 | 1.0 × 10⁴ | 6.9 |
| Run 2a** | 30 min | D6 | 2.0 × 10⁴ | 8.2 |
| Run 2b** | 10 to 30 sec | D6 | 1.0 × 10³ | 7.7 |
| Run 3 | 1 min | D6 | 5.9 × 10² | 5.9 |
| VP2 | | | | |
| From Run 1 (VP1) | 10 to 30 sec | D6 | Negative*** | 7.6 |
| From Run 2b (VP1) | 10 to 30 sec | D7 | Negative*** | 6.6 |
| VP3 | | | | |
| From Run 1 (VP2) | 10 to 30 sec | D7 | Negative*** | 7.5 |

*ffu is for foci-forming units
**The Rotavirus seed used to infect Vero cells in Run 2 (a and b) comprised 6.2 × 10¹⁰ DNA copies of PCV-1/ml. Its titer was 8.2 log CCID50/ml
***Negative means that no fluorescence signal was detected during the Q-PCR

### Results

In Run 1 where a virus contact time of 10 to 30 seconds was used with a MOI of 10⁻¹, a decrease of 2 to 3 logs of PCV-1 DNA content after one virus passage (see VP1) was observed, as compared with the control where the virus solution was not removed but maintained for 6 or 7 days before harvesting the Rotavirus. And it was observed an absence of detectable PCV-1 at the second virus passage (see VP2) using the virus harvest collected after the first virus passage. Noteworthy, the rotavirus titer remained mostly the same, as compared with the control where the virus solution was not removed but maintained for 6 or 7 days before harvesting the Rotavirus (see the last column—Rotavirus titer). The PCV-1 DNA level remained undetectable after a third virus passage using the virus harvest collected after the second virus passage of the Run 1 (see VP3), while the Rotavirus titer was further increased, as compared to the titer obtained after the second virus passage.

In Run 2, it was observed that using 10 to 30 seconds of virus contact time resulted in a 7 logs decrease of the PCV-1 DNA content, as compared to the level of PCV-1 DNA present in the Rotavirus seed. Using a 30 minutes virus contact time still provided good decrease results, as a 6 logs reduction was observed, as compared to the level of PCV-1 DNA present in the Rotavirus seed. Moreover a longer virus contact time, such as 30 minutes, seems to provide a rotavirus with a higher titer (compare 8.2 versus 7.7 values in VP1 row, Run 2 experiment).

2.2 Two additional independent experiments were conducted (Run 4 and Run 5) to assess the effect of using short virus contact times, such as 1 minute, 10 minutes and 30 minutes. The Run 4 experiment included also the assessment of 2 additional series of conditions (see below section 3.2): (i) for a given 30 min virus contact time, compare 10⁻¹ MOI versus 0.5 MOI; (ii) for a given 30 minutes contact time and a given 10⁻¹ MOI, compare using 7.5 μg/ml of trypsin in the virus production medium (see section 1.4) versus 15 μg/ml of trypsin.

Vero cells were seeded at the density of 50,000 cells/cm² and grown for 6 days (Run 4) or 7 days (Run 5) before virus infection. A Rotavirus solution was specifically activated in DMEM supplemented with 20 μg/ml of trypsin for 30 min at room temperature. Before infection, cells were washed with DMEM. They were then infected by contacting them with 10⁻¹ of the above activated Rotavirus solution diluted in DMEM supplemented with 15 μg/ml of trypsin for 1 minute, 10 minutes or 30 minutes. After the indicated contact time, the Rotavirus solution was removed and cells were washed twice with DMEM and further incubated in DMEM supplemented with 7.5 μg/ml of trypsin (Run 4) or with 15 μg/ml of trypsin (Run 5) for 7 days, at which time the virus-containing supernatant was harvested. The virus-containing supernatants were then clarified by centrifugation at 180 g for 10 minutes at room temperature. A sample of virus-containing supernatant (before and after clarification) was subject to a titration assay allowing to measure rotavirus titer and to a Q-PCR assay allowing to determine the PCV-1 DNA content, as described in sections 1.6 and 1.7, respectively. The results are presented in Table 4. They are expressed in log CCID50/ml and DNA copies/ml, respectively.

The same Rotavirus seed was used to infect cells in Run 4 and Run 5, which seed has a titer of 7.5, as measured in log CCID50/ml. Also, a Q-PCR assay for measuring the PCV-1 DNA content performed on a sample of this Rotavirus seed indicated that the PCV-1 DNA was present within this seed at the level of 4×10⁷ DNA copies /ml. This value serves as control value for assessing the DNA PCV-1 reduction when using the method of the invention as exemplified in this section.

TABLE 4
Effect of reducing virus contact time with the
cells on rotavirus titer and PCV-1 DNA content

| | | | PCV-1 | | Rotavirus titer | |
|---|---|---|---|---|---|---|
| | | | (DNA copies/ml) | | (log CCID50/ml) | |
| | | | Before | After | Before | After |
| | Contact | Harvest | Clarifi- | Clarifi- | Clarifi- | Clarifi- |
| MOI 10⁻¹ | time | Day | cation | cation | cation | cation |
| Run 4 | 1 min | D7 | 240 | — | 4.5 | — |
| | 10 min | D7 | Negative | — | 5.4 | — |
| | 30 min | D7 | 230 | — | 5.9 | — |
| Run 5 | 1 min | D7 | 207 | 609 | 5.3 | 5.0 |
| | 10 min | D7 | 554 | 368 | 5.9 | 5.7 |
| | 30 min | D7 | 4513 | 682 | 6.0 | 6.1 |

| | | | | |
|---|---|---|---|---|
| Control | N/A | N/A | 4 × 10⁷ | 7.5 |

### Results

If considering the data obtained in Run 4, the PCV-1 DNA reduction achieved is around 5 logs or more, whether using 1 min, 10 min, or 30 minutes of virus contact time, indicating that any of those short contact times provide good PCV-1 DNA reduction. If considering the data obtained in Run 5, a similar 5 log reduction was observed when using 1 min and 10 min of virus contact time, while a 30 min virus contact time allowed a lower reduction, as a 4 log reduction was observed at this contact time (see the data obtained before clarification). The rotavirus titration, in both Run 4 and Run 5 showed that increasing the virus contact time tends to increase the Rotavirus titer.

### Example 3

### Effect of Varying Rotavirus MOI

3.1 In a separate experiment, the impact of a reduced MOI, i.e. lower than 10⁻¹, such as 10⁻³, 10⁻⁴ and 10⁻⁵ on the PCV-1 DNA content has been assessed.

PCV-1-free Vero cell cultures were grown in VP-SFM either in T-175 flasks or in T-25 flasks. Cells were washed with DMEM, and they were contacted with a Rotavirus solution (diluted in DMEM containing 15 μg/ml of trypsin) at a MOI of 10⁻¹ or 10⁻³ for 10 to 30 seconds or for 30 minutes. In a separate experiment, 10⁻⁴ or 10⁻⁵ MOI of a Rotavirus solution was contacted with washed cells for 10 to 30 seconds or for 30 minutes. Infection with MOI 10⁻¹ and 10⁻³ were tested each in one T-175 flask, while MOI 10⁻⁴ and 10⁻⁵ were tested in T-25 flasks in five culture replicates. Prior to contacting the cells with the virus, the virus was specifically activated as described in section 1.2 in DMEM supplemented with 15 μg/ml of trypsin.

After either 10 to 30 seconds or 30 minutes of virus contact time at 37° C., the virus solution was removed. The cell layer was washed two times with DMEM. Then, DMEM supplemented with 7.5 μg/ml of trypsin was added and the cells were further incubated at 37° C. for 5 or 6 days. Virus-containing supernatants were thus harvested after 5 or 6 days (as indicated in Table 5) and frozen at −70° C.

Based on the same method, a second virus passage (VP2) was done on Vero cells prepared similarly to the above first passage. A virus sample from the above-harvested supernatant arising from the above first virus passage (wherein the virus infection was performed with a MOI of 10⁻³) was thawed and specifically activated as described in section 1.2 in DMEM supplemented with 15 μg/ml of trypsin. After washing the cells with DMEM, cells were infected by contacting them with 10⁻¹ MOI of the activated virus solution (diluted in DMEM medium supplemented with 15 μg/ml of trypsin). After a contact time of 10 to 30 seconds, the virus solution was removed and cells were washed with DMEM two times and further incubated at 37° C. for 7 days in DMEM supplemented with 7.5 μg/ml of trypsin. Then, virus-containing supernatant was harvested and stored at −70° C.

All of the above virus-containing supernatants were subject to a titration assay allowing to measure Rotavirus titer and to a Q-PCR assay allowing to determine the PCV-1 DNA content, as described in section 1.5 and in section 1.7, respectively. The results are presented in Table 5. They are expressed in log ffu/ml and DNA copies/ml, respectively. The Rotavirus seed used to infect Vero cells in the experiment where 10⁻¹ and 10⁻³ MOI of virus were tested comprised 6.2×10¹⁰ DNA copies of PCV-1/ml. This value serves as a control value for assessing the DNA PCV-1 reduction when using the method of the invention as exemplified in this section.

TABLE 5
Effect of reducing rotavirus MOI on rotavirus titer and PCV-1 DNA
content

| Virus | | | PCV-1 | |
|---|---|---|---|---|
| passage | Contact | Harvest | (DNA | Rotavirus titer |
| (VP) | time | day | copies/ml) | (log ffu/ml) |
| VP1 | | | | |
| MOI 10⁻¹ | 10-30 sec | D5 | 1 × 10³ | 6.2 |
| MOI 10⁻³ | 30 min | D5 | Negative | 5.8 |
| *MOI 10⁻⁴ | 10-30 sec | D6 | 5/5 | 3.4, 3.5, 3.6, 3.8, 4.0 |
| | | | Negative*** | |
| *MOI 10⁻⁴ | 30 min | D6 | **4/5 Neg, | 3.2, 3.3, 3.3, 3.5, 3.6 |
| | | | 4.5 × 10⁻² | |
| *MOI 10⁻⁵ | 10-30 sec | D6 | **4/5 Neg, | 2.9, 2.9, 3.0, 3.1, 3.3 |
| | | | 1.5 × 10⁻² | |
| *MOI 10⁻⁵ | 30 min | D6 | 5/5 | 2.8, 2.8, 2.9, 2.9, 2.9 |
| | | | Negative*** | |
| VP2 | | | | |
| MOI 10⁻¹ | 10-30 sec | D7 | Negative*** | 6.7 |
| (from MOI | | | | |
| 10⁻³ VP1) | | | | |

*The experiment at those MOI has been performed in 5 replicates
**4/5 Neg indicate that the Q-PCR assay provided negative results on 4 replicates out of the 5 total ones, the fifth value being specified
***Negative means that no fluorescence signal was detected during the Q-PCR

### Results

Using a MOI lower than 10⁻¹, such as 10⁻³, with a virus contact time of 30 min, provides negative results as far as the PCV-1 DNA content is concerned after one virus passage (VP1) and a Rotavirus titer of 5.8 logs. In the experiment where lower MOI were tested, i.e. 10⁻⁴ and 10⁻⁵, with a virus contact time of 30 minutes or of 10-30 sec, the rotavirus titer obtained was significantly lower, 54.0 log for a MOI of 10⁻⁴ and 53.3 for a MOI of 10⁻⁵. No

PCV-1 DNA (or a value very close to the limit of detection) was detected by Q-PCR following the first virus passage at those low MOI. The PCV-1 negative data were confirmed after a second virus passage using MOI 10⁻¹ of the virus produced during the first virus passage (with a MOI of 10⁻³) and a contact time of 10 to 30 seconds (see VP2 row), while rotavirus titer was increased to 6.7 logs (to be compared with 5.8 logs obtained after the first virus passage). Those results indicate that using lower virus MOI, such as 10⁻³, 10⁻⁴ and 10⁻⁵, also allowed to greatly reduce the PCV-1 DNA content, as compared with results obtained when using 10⁻¹ MOI. As far as the Rotavirus titer is concerned, those lower virus MOI resulted in a significantly lower titer than that obtained when using 10⁻¹ MOI. However, further passaging the virus obtained allowed to further increase the Rotavirus titer, while maintaining the PCV-1 DNA content negative.

3.2 As indicated in section 2.2, the Run 4 experiment was divided into 3 parts based on the conditions which were assessed, the first part having been described in section 2.2.

The second part was aimed at testing the impact of increasing the MOI number on the PCV-1 DNA content: 10⁻¹ versus 0.5.

The third part was aimed at testing the impact of using 15 μg/ml of trypsin in the virus production medium (see section 1.4) versus using 7.5 μg/ml of trypsin as in previous experiments.

PCV-1-free Vero cell cultures were grown in VP-SFM in cell factories as described in section 1.1. The experimental conditions were identical to the conditions described in section 2.2. A sample of all the virus-containing supernatants was subject to a titration assay allowing to measure rotavirus titer and to a Q-PCR assay allowing to determine the

PCV-1 DNA content, as described in sections 1.6 and 1.7, respectively. The results are presented in Table 6. They are expressed in log CCID/ml and DNA copies/ml, respectively. The Rotavirus seed used to infect cells has a titer of 7.5 as measured in log CCID50/ml. A Q-PCR assay for measuring the PCV-1 DNA content performed on a sample of this Rotavirus seed indicated that the PCV-1 DNA was present within this seed at the level of 4×10⁷ DNA copies /ml.

TABLE 6
Effect of increasing rotavirus MOI on rotavirus titer and PCV-1 DNA
content

| | | | | Rotavirus titer |
|---|---|---|---|---|
| | Contact | Harvest | PCV-1 | (log |
| | time | day | (DNA copies/ml) | CCID50/ml) |
| 1. MOI 10⁻¹ | 30 min | D7 | 2.3 × 10² | 5.9 |
| Trypsin 7.5 μg/ml | | | | |
| 2. MOI 10⁻¹ | 30 min | D7 | 2.1 × 10² | 6.8 |
| Trypsin 15 μg/ml | | | | |
| 3. MOI 0.5 | 30 min | D7 | 1.9 × 10⁴ | 7.2 |
| Trypsin 15 μg/ml | | | | |
| Control | N/A | N/A | 4 × 10⁷ | 7.5 |

### Results

Using a virus contact time of 30 minutes with a trypsin concentration of 15 μg/ml in the virus production medium resulted in a good reduction of the PCV-1 DNA content at both MOI 10⁻¹ and MOI 0.5 (compare rows 2 and 3). However, using MOI 10^{31 1} resulted in a better reduction (5 logs reduction), as compared to MOI 0.5 (3 logs reduction). With respect to the Rotavirus yield, titration data indicated that using more MOI (0.5 versus 10⁻¹) tends to give a higher Rotavirus titer (compare rows 1 and 2 with row 3). As far as the trypsin concentration in the virus production medium is concerned, the data presented in Table 6 (rows 1 and 2) indicated that more trypsin during production (15 μg/ml) allows to increase the titer of almost one log.

### Example 4

### Optimization of the Conditions—Preparation of a Rotavirus Pre-Master Seed

4.1 PCV-1-free Vero cell cultures were grown in Cell Factories in VP-SFM so as to reach a cell density of 250,000 cells/cm².They were then washed with DMEM. A rotavirus solution was specifically activated in DMEM supplemented with trypsin at a concentration of 20 μg/ml during 30 minutes at room temperature. After washing them with DMEM, cells were then infected by contacting them with 10⁻¹ MOI of the activated rotavirus solution (diluted in DMEM containing 15 μg/ml of trypsin) for 10 minutes at 37° C. After a 10 minutes contact time, the virus solution was removed. The cell layer was washed two times with DMEM. Then, DMEM supplemented with 15 μg/ml of trypsin was added to the cells and the cells were further incubated at 37° C. for 7 days, at which time the virus-containing supernatant was harvested. The viral harvest was then clarified by centrifugation at room temperature at 1000 rpm. The viral supernatant was collected and stored at −70° C.

The above virus-containing supernatant was subject to a titration assay allowing to measure rotavirus titer and to a Q-PCR assay allowing to determine the PCV-1 DNA content, as described in sections 1.6 and 1.7, respectively. The results are presented in Table 7. They are expressed in log ffu/ml and DNA copies/ml, respectively.

TABLE 7
Effect of a 10 minutes virus time contact on rotavirus titer and PCV-1
DNA content

| | Contact | Harvest | PCV-1 | Rotavirus titer |
|---|---|---|---|---|
| | time | day | (DNA copies/ml) | (log ffu/ml) |
| Virus passage | 10 min | D7 | 950 | 6.4 |
| MOI 10⁻¹ | | | | |
| Control* | N/A | N/A | 6 × 10⁷ | 7 |

*The virus seed used to inoculate the cells was titrated in log ffu/ml and was subject to a Q-PCR assay for measuring the PCV-1 DNA content present in the seed. Therefore, those values serve as control values of the experiment

### Results

Starting from a Rotavirus seed contaminated with 6×10⁷ PCV-1 DNA copies/ml (see the Control row and the PCV-1 column), the method of the invention as exemplified in the present Example 4, i.e. combining a virus MOI of 10⁻¹ and a virus reduced contact time with the cells of 10 minutes, allowed to achieve a reduction of the PCV-1 DNA content greater than 5 logs, while the rotavirus titer is not significantly impacted, as the virus loss is no more than 0.6 log. After one virus passage according to the method of the invention, as exemplified in the present Example 4, the DNA content of PCV-1 was reduced by more than 99.99%, as compared to the initial level present in the Rotavirus seed.

4.2 The Rotavirus pre-Master seed obtained according to the above was also submitted to a PCV-1 infectivity assay. A sample of the above pre-Master seed was first neutralized with an anti-Rotavirus antibody and then inoculated on Vero cells, as described in section 1.8. 14 days after the inoculation (see D14 in Table 8), inoculated Vero cells were collected and RNA was extracted as described in section 1.8, so that a RT-Q-PCR aimed at amplifying specific mRNA from PCV-1 could be performed as described in section 1.8. The results of this RT-Q-PCR are presented in Table 8. As a negative control, in particular to check that the Vero cells used in this assay are PCV-1 free, a RT-Q-PCR was performed also on Vero cells before inoculating them with the pre-Master seed (see RT-Q-PCR DO in Table 8).

TABLE 8
Test of PCV-1 infectivity on the pre-Master seed by measuring the PCV-1
mRNA load

| | RT-Q-PCR | RT-Q-PCR |
|---|---|---|
| | D0 | D14 |
| Rota Pre-Master | Negative* | Negative* |
| seed | | |

*Negative means that no fluorescence signal was detected during the Q-PCR

### Results

The negative results of Table 8 obtained at D14 indicate that the Rotavirus pre-Master seed obtained according to the method of the invention as exemplified in the present example did not contain any infectious PCV-1 particles.

1. A process for producing a virus of interest in cell culture, comprising contacting a population of cells with a solution comprising the virus of interest, wherein the cells are susceptible to infection with the virus of interest, characterised in that the contact time between the solution comprising the virus of interest and susceptible cells is inferior than or equal to 120 minutes.

2. A process for producing a virus of interest comprising the steps of:
a) contacting a population of cells with a solution comprising the virus of interest for a period inferior than or equal to about 120 minutes, wherein the cells are susceptible to infection with the virus of interest;
b) removing the solution comprising the virus of interest from the cells, and
c) incubating the cells in a culture medium to produce a population of replicated virus of interest.

3. The process of claim 2 further comprising the steps step of:
d) formulating the produced virus with a suitable pharmaceutical carrier

4. The process according to claim 1, wherein the solution comprising the virus of interest comprises one or more adventitious agents.

5. The process according to claim 4, wherein the cells are susceptible to infection with one or more of the adventitious agents.

6. A process for producing a virus of interest comprising the steps of:
a) contacting a population of cells with an initial solution comprising the virus of interest and at least one adventitious agent, wherein the cells are susceptible to infection with the virus of interest and the contact period of time is sufficient to permit infection of at least a subset of the population of cells with the virus of interest,
b) removing the solution comprising the virus of interest and the at least one adventitious agent from the cells,
c) incubating the cells in a culture medium to produce a population of replicated virus of interest,
wherein the DNA content level of the at least one adventitious agent is reduced by at least 90% in the population of replicated virus of interest as compared to the level present in the initial solution comprising the virus of interest and the at least one adventitious agent.

7. The process of claim 6 wherein the at least one adventitious agent is removed or substantially removed from the population of replicated virus of interest as compared to the initial solution comprising the virus of interest and the at least one adventitious agent.

8. The process according to claim 6, wherein the contact period of time is inferior than or equal to 120 minutes.

9. (canceled)

10. The process according to claim 6, wherein the contact time in step a) is sufficient to permit adsorption of the virus of interest to at least a subset of the population of cells.

11. (canceled)

12. The process according to claim 6, wherein the cells are susceptible to infection with one or more of the adventitious agents.

13. The process according to claim 2, wherein the series of steps a) to c) is repeated at least once, using the population of replicated virus of interest obtained at step c) as the solution comprising the virus of interest for contacting the population of cells according to the further step a).

14. The process according to claim 1, wherein the contact period of time time is ranging from 10 seconds to 120 minutes, from 1 minute to 30 minutes, from 5 minutes to 15 minutes, is 10 minutes.

15. The process according to claim 1, wherein the cells are mammalian, avian or insect cells.

16. The process according to claim 1, wherein the cells are selected from the group: MRC-5, Vero, CHO, FRHL-2, MDCK, PER.C6, EBx® cells and Chicken Embryo Fibroblasts [CEF].

17. The process according to claim 16, wherein the cells are Vero cells.

18. The process according to claim 1, wherein the virus of interest is an attenuated virus.

19. The process according to claim 1, wherein the virus of interest is selected from the group consisting of: measles, mumps, rubella, human papilloma virus (HPV), Baculovirus, influenza, varicella zoster virus (VZV), poliomyelitis virus, Epstein bar virus (EBV), Human Immunodeficiency virus (HIV), Herpes simplex virus (HSV), Hepatitis B (HBV), Hepatitis C (HCV), Hepatitis E (HBV), Dengue virus, cytomegalovirus (CMV) respiratory syncytial virus (RSV), rabies virus, human Rotavirus (HRV), adenovirus or Hepatitis A (HAV). CHECK MARKUSH GROUP FORM

20. The process according to claim 19, wherein the virus of interest is human Rotavirus (HRV).

21. The process according to claim 1, wherein the cells are contacted with the virus of interest at a multiplicity of infection (MOI) of about 1, about.

22. The process according to claim 4, wherein the adventitious agent is a virus, selected from the group consisting of, PCV, PCV-1 and PCV-2.

23. The process according to claim 2, wherein the cells in step c) are incubated for about 1 to 30 days.

24. The process according to claim 2, wherein the cells in step c) are incubated at a temperature between about 25° C. and about 41° C.

25. The process according to claim 2, further comprising the step of collecting the population of replicated virus of interest.

26. The process according to claim 2, further comprising the step of clarifying the population of replicated virus of interest to remove cell debris.

27. The process according to claim 2, further comprising the step of inactivating the virus of interest in the population of replicated virus of interest.

28. The process according to claim 2, further comprising the step of sterile filtering the population of replicated virus of interest through a sterile grade filter membrane.

29. A virus obtainable by a process according to claim 2.

30. An immunogenic composition produced from the virus obtained according to claim 29.

31. (canceled)

32. (canceled)

33. The immunogenic composition of claim 30 wherein the composition is substantially free of an adventitious agent.

34. The immunogenic composition according to claim 33, wherein the virus is HRV.

35. The immunogenic composition or vaccine according to claim 33 wherein the composition is substantially free of PCV-1 or PCV-2.

36. (canceled)

37. (canceled)
